(19) United States
(12) Patent Application Publication
(10) Pub. No.: US 2023/0030675 A1
(43) Pub. Date: Feb. 2, 2023

(54) SOLID STATE ANTIMICROBIAL COMPOSITIONS AND METHODS FOR PRODUCING AND USING SAME

(71) Applicant: Armis Biopharma, Inc., Fort Collins, CO (US)

(72) Inventors: Scott Noblitt, Fort Collins, CO (US); Edwin D. Neas, Nunn, CO (US)

(21) Appl. No.: 17/758,452

(22) PCT Filed: Jan. 7, 2021
(86) PCT No.: PCT/US21/12536

§ 371 Date: Jul. 6, 2022

Related U.S. Application Data
(63) Continuation-in-part of application No. 16/736,546, filed on Jan. 7, 2020.
Application No. 16/736,546 is a continuation-in-part of application No. 16/736,546, filed on Jan. 7, 2020.
Application No. 16/736,546 is a continuation-in-part of application No. PCT/US18/41163, filed on Jul. 7, 2018.
(60) Provisional application No. 62/530,045, filed on Jul. 7, 2017.

(51) Int. Cl. A61K 31/327; A61K 31/336; A61K 33/06; A61K 9/00; A61P 17/02

(52) U.S. Cl. CPC A61K 31/327; A61K 31/336; A61K 33/06; A61K 9/0014; A61P 17/02

(57) ABSTRACT

The present invention relates to antimicrobial, disinfecting, and wound healing compositions and methods for producing and using the same. The compositions may contain a peroxyacid, a hydroperoxide, the parent carboxylic acid of the peroxyacid, and a salt.

### CROSS-REFERENCE TO RELATED APPLICATION

The present application claims priority under 35 U.S.C. § 120 to U.S. application Ser. No. 16/736,546 filed Jan. 7, 2020, which is a Continuation In Part of International Patent Application No. PCT/US2018/041163, filed Jul. 7, 2018, which claims priority under 35 U.S.C. § 119(e) to U.S. Provisional Application Ser. No. 62/530,045 filed Jul. 7, 2017, the disclosures of which are incorporated by reference herein in their entirety.

### FIELD OF THE INVENTION

The present invention relates to solid state antimicrobial, disinfecting, and wound healing compositions and methods for producing and using the same. The compositions contain a peracid, a hydroperoxide, the parent carboxylic acid of the peroxyacid, and a salt.

### BACKGROUND OF THE INVENTION

Human and mammalian health is impacted by the spread of microbial organisms such as viruses, bacteria, and fungi. Microbial organisms continue to cause a variety of sicknesses and ailments. In the wake of widespread microbial organism infections, the public has become even further concerned with sanitization, both of person and property. Consequently, there has been an extensive research on the development of suitable antimicrobial compositions, in particular of antimicrobial compositions that provide immediate and residual kill of microbial organisms.

Currently, there exist several compositions and methods for reducing and/or eliminating microbial organisms from various surfaces. Conventional antimicrobial cleansing products such as hard surface cleaners and surgical disinfectants are typically formulated to provide bacteria removal during washings. Only a few such products have been shown to provide a residual effectiveness against Gram-positive bacteria; however, even such compositions provide only limited residual effectiveness against Gram-negative bacteria. By “residual effectiveness,” it is meant that the subject antimicrobial controls microbial growth on a substrate by either preventing growth of microbes or engaging in continuous kill of microbes for some period of time following the washing and/or rinsing process. There are a myriad of solutions available that claim to kill 99.9% of MRSA and other vegetative bacteria and some spores on surfaces and skin (e.g., hand sanitizers). Therefore, these solutions leave one viable bacterium, or spore, in a thousand or a thousand viable bacteria, or spores, in a million after treatment. However, contaminated surfaces can contain millions of bacteria, some of which can be contained within complex matrices such as blood drops, thus making them difficult to kill. Other types of bacteria, such as Bacillus subtilis, form biofilms on surfaces of endoscopes and other medical devices for insertion into the body, which affects the kill efficacy of most disinfectants. These low level disinfectants, often called sanitizers, that claim to kill 99.9% of the bacteria present will not completely kill all bacteria which are present in higher populations (colonized), contained within a complex matrix, or existing as a biofilm.

There also exist antimicrobial compositions for diminishing the growth of bacterial infections in wounds. Wound healing and “good” care of wounds has been synonymous with topical prevention and management of microbial contamination. Today's primary therapy involves the use of either topical application of antiseptics or systemic and topical use of antibiotics. The general perspective is that topical application of antibiotics to wounds has no advantages over the use of other antiseptic methods and may increase the risk of wound-healing by producing a sovereign bacteria that is resistant within the wound. Quite often proper wound healing is impaired with devastating consequences such as severe morbidity, amputations, or death. Sepsis is the leading cause of death after a burn injury. Multiple antibiotic resistant bacteria and fungus now account for the bulk of deaths due to sepsis in burns, the etiology of which is due to antibiotic resistant bacteria and biofilm formation in the wound and extraneous nosocomial infections. Most antiseptics are not suitable for open wounds because they may impede wound healing by direct cytotoxic effects to keratinocytes and fibroblasts. In general, current topical antiseptics have limited bactericidal effect (e.g., 3 log reduction in 30 minute exposure) and nearly all have some cytotoxicity effect which varies with concentration and application time.

Peracetic acid has been applied for purposes such as disinfecting medical supplies, preventing biofilm formation in pulp industries, disinfecting during water purification, and plumbing disinfection. Peracetic acid is produced by a reaction between hydrogen peroxide and acetic acid or it can also be produced by oxidation of acethaldehyde. Peracetic acid is a very powerful oxidant; the oxidation potential outranges that of chlorine and chlorine dioxide. Peracetic acid is not known to be involved in any significant cellular metabolism and is typically produced with toxic sulfuric acid catalyst. A drawback of the peroxyacid-based chemical disinfectants is their inherent lack of stability, which poses a challenge for shelf-life when used for long term applications. Thus, a need exists for a peracid-based disinfectant, which is an effective broad spectrum antimicrobial, is in an easily removable homogenous antimicrobial coating composition providing both short-term and extended long-term antimicrobial efficacy after application to a surface or a wound. In addition, there is a continuing need for new topical wound sanitizers, healers or both, and in particular there is a need to develop peroxyacids that are effective sporocides, bactericids and virucides for wounds which are easy to handle and store. Moreover, there is a need for peroxyacids that are easy to handle and store and that have a low corrosive nature. It is therefore desirable to develop a sanitizer that does not decompose rapidly and violently and that can be used as a topical wound sanitizer or as an antimicrobial coating.

### SUMMARY OF THE INVENTION

In some aspects, the present invention relates to novel antimicrobial, disinfecting, and/or wound healing compositions and methods for producing and using the same. The compositions contain a peracid, a hydroperoxide, the parent carboxylic acid of the peroxyacid, and a salt.

In a first aspect, the present disclosure provides a solid-state antimicrobial composition containing a peroxyacid, a hydroperoxide, the parent carboxylic acid of the peroxyacid, and a salt selected from a lithium salt, a sodium salt, a potassium salt, a rubidium salt, a cesium salt, a zinc salt, a magnesium salt, a calcium salt and a combination thereof. In some embodiments, the hydroperoxide is hydrogen peroxide. In some embodiments the salt is a magnesium salt. In some embodiments, the magnesium salt is selected from a magnesium oxide, magnesium hydroxide, magnesium carbonate, magnesium acetate tetrahydrate, and a combination thereof. In some embodiments, the magnesium salt is magnesium acetate tetrahydrate. In some embodiments the peroxyacid is peracetic acid. In some embodiments, the peroxyacid is a magnesium salt of the peroxyacid. In some embodiments the composition further contains at least one of a bis(hydroperoxide) or an epoxide. In some embodiments, the peroxyacid is peracetic acid and the bis(hydroperoxide) is 3,3-bis(hydroperoxy)butanoic acid. In some embodiments, the composition is formulated as a wound healing composition. In some embodiments, the wound healing composition is formulated as a gel, a liquid, lotion, skin patch, irrigation gel, a spray, application granules, or a combination thereof.

In a second aspect, the present disclosure provides a method for treating a wound infection in a subject comprising contacting the infected wound in the subject with a therapeutically effective amount of the composition of the first aspect. In some embodiments, the infected wound is a surgical wound, battle wound, accidental wound, thermal burn wound, chemical burn wound, chronic wound, decubitus ulcer, foot ulcer, venous ulcer, laser treatment wound, sunburn, or an abrasion. In some embodiments, the infected wound is contacted with the composition once a day. In some embodiments, the composition is formulated as a gel, a liquid, lotion, skin patch, irrigation gel, a spray, application granules, or a combination thereof.

### BRIEF DESCRIPTION OF THE FIGURES

The accompanying drawings, which are incorporated herein and constitute part of this specification, illustrate the presently preferred embodiments of the invention, and, together with the general description above and the detailed description given below, serve to explain the features of the invention. In the drawings:

FIG. 1 is a graphic illustration of phases of wound healing.

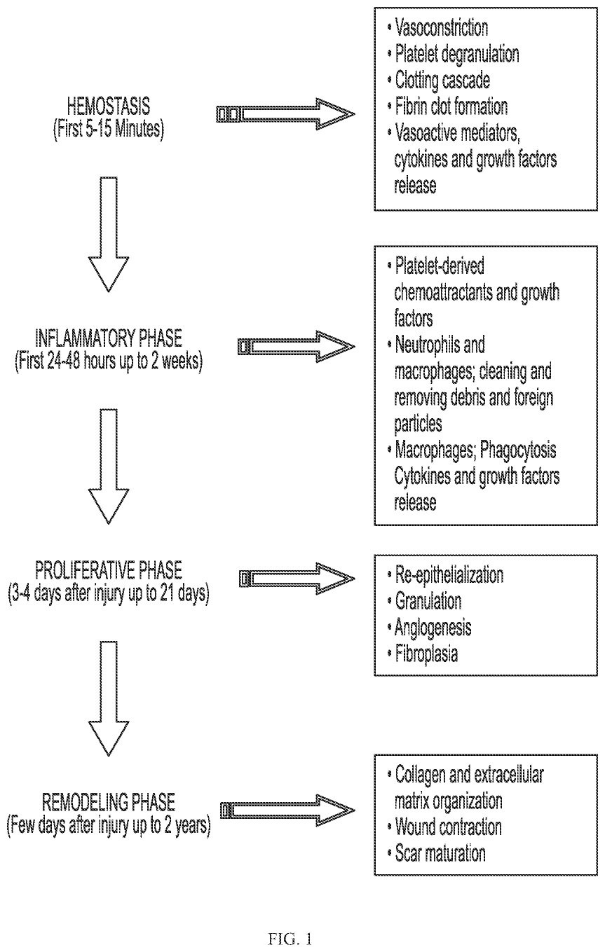

FIG. 2 is a schematic illustration of inflammatory phases of wound healing.

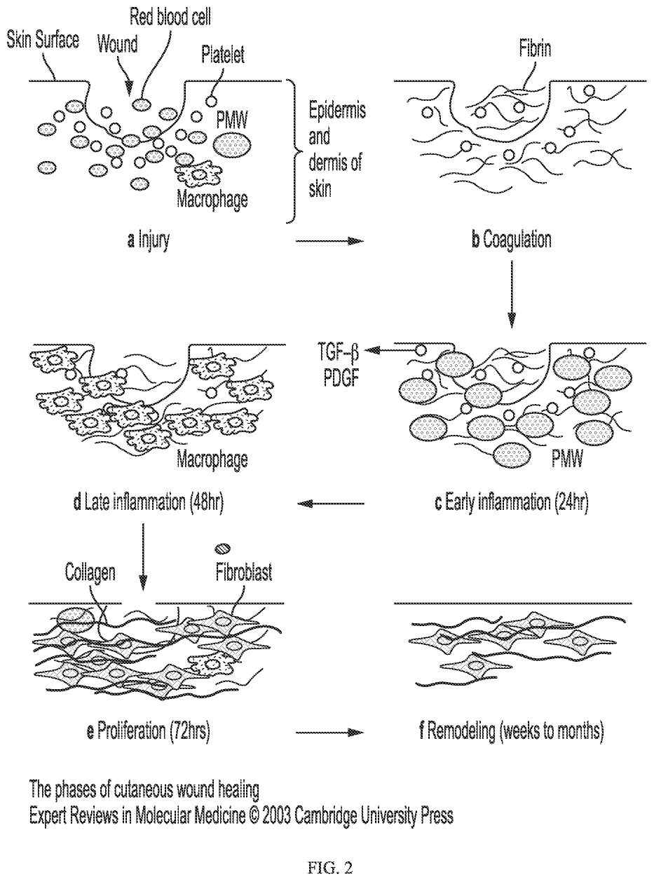

FIG. 3 is a reaction scheme for a reaction comprising pyruvic acid and hydrogen peroxide according to an embodiment of the present invention.

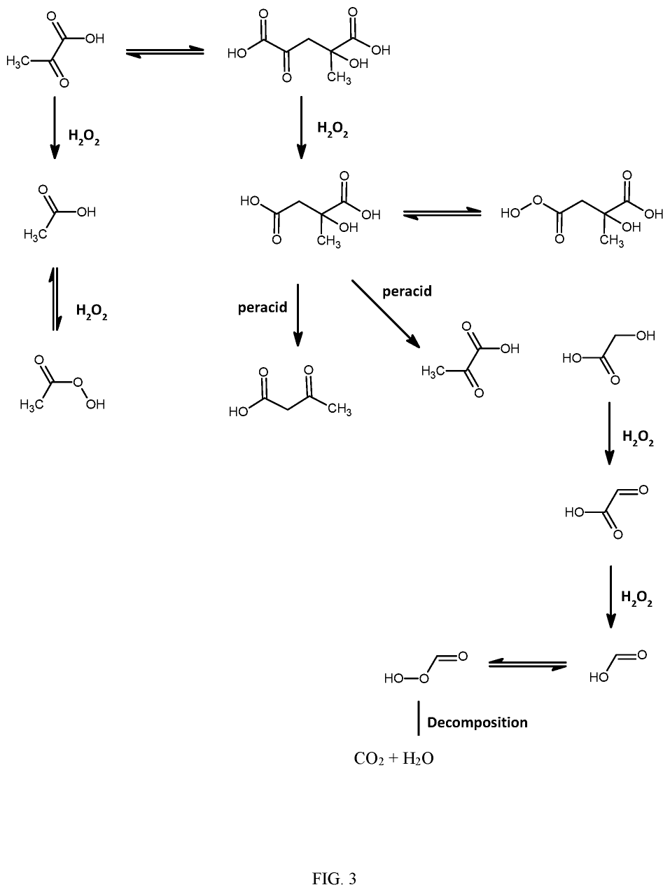

FIG. 4 is a reaction scheme for a reaction comprising acetoacetic acid and hydrogen peroxide according to an embodiment of the present invention.

FIG. 5 is a reaction scheme for a reaction comprising maleic acid and hydrogen peroxide according to an embodiment of the present invention.

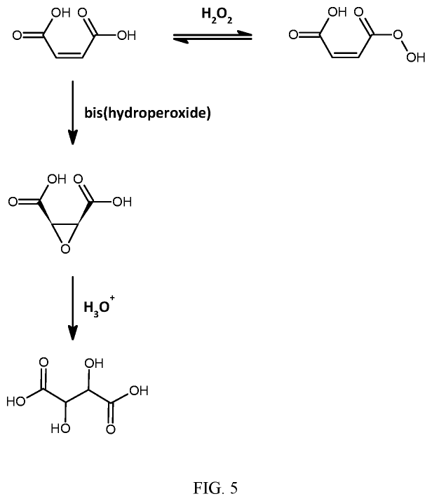

FIG. 6 is a reaction scheme for a reaction comprising citraconic acid and hydrogen peroxide according to an embodiment of the present invention.

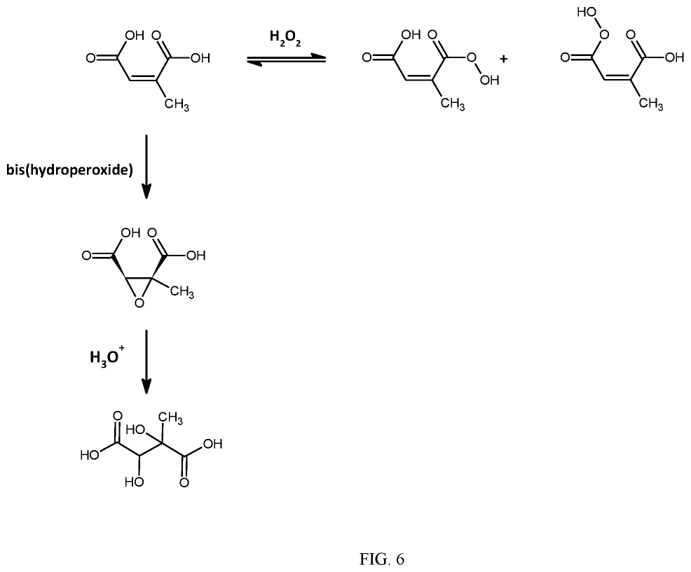

### DETAILED DESCRIPTION OF THE INVENTION

While various aspects and features of certain embodiments have been summarized above, the following detailed description illustrates a few embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers expressing quantities of ingredients, dimensions, reaction conditions, and so forth, used in the specification and claims, are to be understood as being modified in all instances by the term “about”.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

For clarity, terms used herein are to be understood as described herein or as such term would be understood by one of ordinary skill in the art of the invention. Additional explanation of certain terms used herein, are provided below:

“wt %” refers to the weight percent relative to the total weight of the solution or dispersion.

“Microorganism” is meant to include any organism comprised of the phylogenetic domains of bacteria and archaea, as well as unicellular (e.g., yeasts) and filamentous (e.g., molds) fungi, unicellular and filamentous algae, unicellular and multicellular parasites, viruses, virinos, and viroids.

“Film-forming agent” or “water soluble or water dispersible coating agent,” which may be used interchangeably herein, refer to agents that form a film and are employed to provide protective coating to the surface of interest. These agents are either water soluble or water dispersible. These agents are described in further detail below.

“Antimicrobial agent” as used herein refers to a compound or substance having antimicrobial properties

“Biocide,” as used herein, refers to a chemical agent, typically broad spectrum, which inactivates or destroys microorganisms. A chemical agent that exhibits the ability to inactivate or destroy microorganisms is described as having “biocidal” activity.

“Biofilm” refers to a structured community of microorganisms encapsulated within a self-developed polymeric matrix and adherent to a living or inert surface. “Drying” refers to a process by which the inert solvent or any other liquid present in the formulation is removed by evaporation.

“Disinfectant” as used herein is a chemical that kills 99.9% of the specific test microorganisms in 10 minutes under the conditions of the test. (Germicidal and Detergent Sanitizing Action of Disinfectants, Official Methods of Analysis of the Association of Official Analytical Chemists, paragraph 960.09 and applicable sections, 15th Edition, 1990 (EPA Guideline 91-2)).

“Sterilization” or “sterilant” as used herein refers to the inactivation of all bio-contamination.

“Locus” as used herein, comprises part or all of a target surface suitable to be coated.

In some aspects, the present invention relates to antimicrobial, disinfecting, and/or wound healing compositions and methods for producing and using the same. The compositions contain a peroxyacid, a hydroperoxide, the parent carboxylic acid of the peroxyacid, and a salt. Some aspects of the present invention provide methods for treating a wound on a subject by contacting the wound with a therapeutically effective amount of the composition.

In general, peroxyacids or peracids are compounds of oxidized form of a base organic acid (generally a carboxylic acid) that exist in equilibrium with an oxidizer (generally hydrogen peroxide) and water. Peracids may be oxidized from other carboxylic acids, e.g. acetic acid, citric acid, succinic acid, short chain fatty acids, etc.

As used herein, “peracid,” “peroxyacid,” “percarboxylic,” and “peroxy-carboxylic acid,” and are used interchangeably herein and refer to compounds generally having the formula R(CO₃H)ₙ, where, for example, R is an alkyl, arylalkyl, cycloalkyl, aromatic, or heterocyclic group, and n is one, two, or three, and named by prefixing the parent acid with “peroxy-.” The R group can be saturated or unsaturated as well as substitut-ed or unsubstituted. Peroxycarboxylic acids can be made by the direct action of an oxidizing agent on a carboxylic acid, by autoxidation of aldehydes, or from acid chlorides, and hydrides, or carboxylic anhydrides with hydrogen or sodium peroxide.

Peroxycarboxylic acids useful in the compositions and methods of the present invention include peroxyformic, peroxyacetic, peroxypropionic, peroxybutanoic, peroxy-pentanoic, peroxyhexanoic, peroxyheptanoic, peroxyoctanoic, peroxynonanoic, peroxy-decanoic, peroxyundecanoic, peroxydodecanoic, or the peroxyacids of their branched chain isomers, peroxylactic, peroxymaleic, peroxyascorbic, peroxyhydroxyacetic, peroxyoxalic, peroxymalonic, peroxysuccinic, peroxyglutaric, peroxyadipic, peroxypimelic and peroxy-suberic acid and mixtures thereof. In some embodiments, the compositions of the invention utilize a combination of several different peroxycarboxylic acids. For example, in some embodiments, the composition includes one or more C1 to C4 peroxycarboxylic acids and one or more C5 to C11 peroxycarboxylic acids. Especially preferred is an embodiment in which the peroxycarboxylic acid is peracetic acid (C2), peroxy propionic acid (C3), peroxybutanoic acid (C4), peroxysuccinic and peroxymalonic acid. It should be noted that both the peroxy-succinic and peroxymalonic acid may come from the alpha-keto dicarboxylic acids. Furthermore, because these acids exist in the Krebs cycle they are metabolically active.

The compositions also include the parent carboxylic acid of the peroxyacid. By “the parent carboxylic acid” is meant the corresponding carboxylic acid from which the peroxyacid is derived or is degraded into under a typical storage or production conditions. For example, in some embodiments, the parent acid is acetic acid, and the peroxyacid is peroxyacetic acid. Suitable parent carboxylic acids include, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, or the acids of their branched chain isomers, lactic acid, maleic acid, ascorbic acid, hydroxyacetic acid, oxalic acid, malonic acid, succinic acid, glutaric acid, adipic acid, pimelic acid and suberic acid and mixtures thereof.

In some embodiments, the parent carboxylic acid is present in the composition of the invention in an amount of about 120.4 mM or less, typically, about 12.4 mM or less, more typically, about 6.2 mM or less, often about 2.5 mM or less, more often, about 1.2 mM or less, still more often about 0.62 mM or less, yet more often about 0.31 mM or less, and most often about 0.062 mM or less.

In some embodiments, the compositions and methods of the present invention include peroxyacetic acid. Peroxyacetic (or peracetic) acid is a peroxycarboxylic acid having the formula: CH₃COOH. Generally, peroxyacetic acid is a liquid having an acrid odor at higher concentrations and is freely soluble in water, alcohol, ether, and sulfuric acid. In some embodiments, the peroxyacid is a magnesium salt of the peroxyacid.

The composition also contains a hydroperoxide, such as for example hydrogen peroxide or an organic peroxide. In some embodiments, the hydroperoxide is hydrogen peroxide. Typically, the amount of hydrogen peroxide present in the wound healing compositions of the invention is about 715 mM or less, typically about 71.5 mM or less, more typically about 35.8 mM or less, often about 14.3 mM or less, more often about 7.2 mM or less, still more often about 3.6 mM or less, yet more often about 1.8 mM or less, and most often about 0.35 mM or less.

In some embodiments, the composition further contains one or more optional compounds, such as for example tartaric acid, formic acid, cis-epoxysuccinic acid, methyltartaric acid, acetic acid, cis-epoxymethylsuccinic acid, maleic acid, citramalic acid, citraconic acid or a combination thereof. In some embodiments, the composition also optionally includes an oxidized acetoacetate compound.

In some embodiments, the composition contains acetoacetic acid. Acetoacetic acid is one of the ketone bodies (along with 3-hydroxybutyric acid and acetone, although acetone is just a byproduct), which are major energy sources for the body, particu-larly during starvation. Ketone bodies are involved in pathways related to the Kreb's cycle, lipogenesis, sterol biosynthesis, glucose metabolism, β-oxidation of fatty acids, mitochondrial electron transport chain, intracellular signal transduction pathways, hormonal signaling, and the microbiome (Cotter, D. G., et al., Am. J. Physiol, Heart Circ. Physiol., 2013, 304, H1060-H1076). It has been tied to skin formation/biosynthesis in rats (Edmond, J., J. Biol. Chem., 1974, 249, 72-80). Additionally, it was just recently found to upregulate osteoblasts and in-crease bone formation (Saito, A., et al. Biochem. Biophys. Res. Comm., 2016, 473, 537-544).

Because acetoacetic acid can be converted into acetyl-CoA in vivo, its ability to affect biological processes is extremely high. However, its presence in the solution is unexpected because acetoacetic acid is an unstable compound that reacts intramolecularly and irreversibly, producing acetone and carbon dioxide. Thus, it is expected to be unstable in all solvents and even as a solid compound.

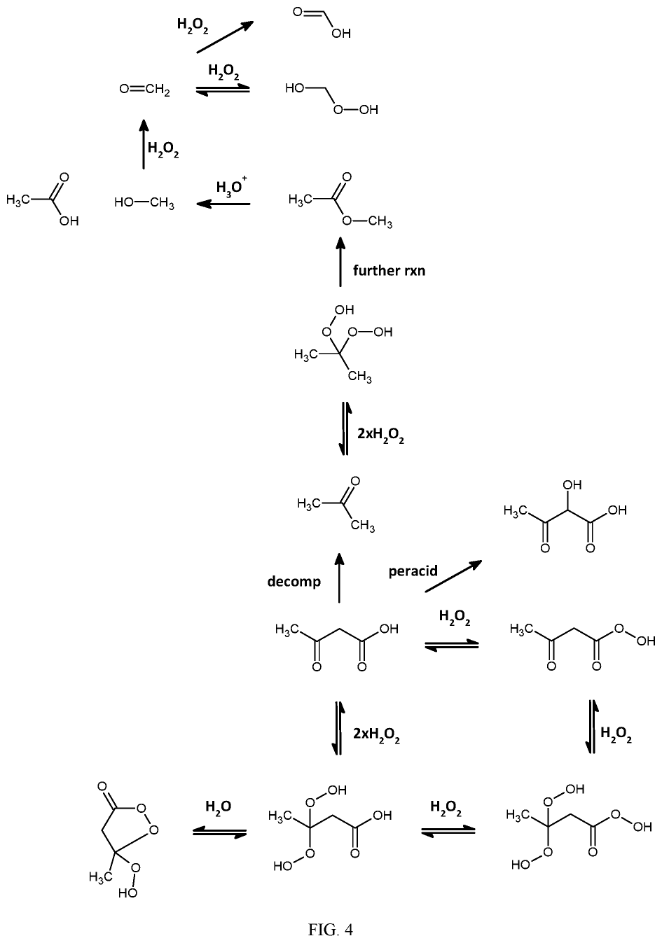

However, acetoacetic acid represents a rather unique case where a compound is stabilized by the addition of hydrogen peroxide, whereas normally the addition of a per-oxide leads to chemical oxidation/degradation. This stabilization is caused by the formation of a range of possible peroxide “adducts” with its ketone functionality and possibly its carboxylic acid. Because both moieties are required for intramolecular “self-destruction”, the formation of these other forms slows down the decomposition of the compound. Peroxide adducts may include 3,3-bis(hydroperoxy)butanoic acid, 3,3-bis(hydroperoxy)butaneperoxoic acid, 3-oxobutane-peroxoic acid, and 5-hydroperoxy-5-methyl-1,2-dioxolan-3-one. This stabilization is shown in the reaction scheme of FIG. 4. The compositions may be further stabiliz-ed by citramalic acid or an acetoacetate ester, such as methyl or ethyl acetoacetate. In some embodiments, the composition also contains a bis(hydroperoxide), or an epoxide, or both. In some embodiments the composition further contains a peroxyacid that is peracetic acid and a bis(hydroperoxide) that is 3,3-bis(hydroperoxy)butanoic acid.

It was particularly unexpected that stable compositions of peracids and bis(hydroperoxides) could be prepared, since peracids are very strong oxidizing agents even at a pH of 2 to 8 because the water soluble peracids are decomposing to form free radicals.

In some embodiments, the compositions may contain peroxycitraconic acid. The peroxycitraconic acid may be either (2Z)-4-hydroperoxy-3-methyl-4-oxobut-2-enoic acid, (2Z)-4-hydroperoxy-2-methyl-4-oxobut-2-enoic acid, or a mixture thereof. In other embodiments, the compositions may comprise diperoxycitraconic acid, i.e., (2Z)-2-methyl-but-2-enediperoxoic acid. In other embodiments, the antimicrobial composition further comprises peroxycitramalic acid. The peroxycitramalic acid may be either 4-hydroperoxy-2-hydroxy-2-methyl-4-oxobutanoic acid, 4-hydroperoxy-3-hydroxy-3-methyl-4-oxobutanoic acid, or a mixture thereof.

The antimicrobial composition is in the solid-phase. In some embodiments, the composition is in the form of a powder. The solid-phase composition provides certain advantages over liquid-phase antimicrobial treatments. One advantage associated with the present invention is that the solid-phase antimicrobial composition is easier to apply and control than a liquid-based composition. In part, this is because the solid-phase composition does not release its antimicrobial components until it comes into contact with moisture, for example from body tissue or fluid. Another advantage is that once applied, the solid-phase composition attaches to the surface of the article, substrate, skin or wound better than liquid compositions. Additionally, the solid form of the composition advantageously enables the slow release of the antimicrobial compounds in the composition, once the compsition comes into contact with fluids and/or tissue, such as for example those on or in a subject's body. The solid-phase composition may be configured so that the release of the antimicrobial compounds within the composition (e.g., peroxycarboxylic acid) is sustained over a predetermined time period of continuous or intermittent exposure to fluids such as bodily fluids. It may also be configured so that the amount and/or concentration of the antimicrobial compound released is above a predetermined threshold for effective antimicrobial effect, either locally, or within a region exposed to the composition. Further, the solid-phase antimicrobial composition is more stable than compositions containing the individual compounds reacted to form the solid-phase antimicrobial composition.

Also, the solid-phase antimicrobial composition is useful for preventing or diminishing the risk of infection even for subjects who have become resistant to antibiotics. The peroxyxcarboxylic acid in the antimicrobial composition is able to breach the cell walls of microbes and destroy the contents of the cell, rendering the microbes incapable of developing resistance to the composition. The solid-phase form of the composition makes it easier to get the antimicrobial composition directly to or near the site of microbes, where it can destroy them.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

The composition can be made by any suitable method. The solid phase antimicrobial composition contains a salt. The solid-phase peroxyxcarboxylic acid salt can be made by mixing hydrogen peroxide, a peroxycarboxylic acid, a salt, to form a solid-phase peroxycarboxylic acid salt precipitate. The reaction to form the solid-phase composition can be performed in a solvent, which may be water, or a lower (water-soluble) alcohol, such as, for example, methanol, ethanol, isopropanol and the like. If the peroxycarboxylic acid is not partially, or completely, miscible in the aqueous reaction solution or suspension, the peroxycarboxylic acid can first be solubilized, for example by pre-dissolving it in an organic solvent. Suitable organic co-solvents include, for example, ethyl acetate, t-butyl alcohol, methanol, tetrahydrofuran, tetrahydropyran, and ethanol and mixtures thereof, with ethyl acetate and t-butyl alcohol being most preferred. Some organic solvents are not preferred because they can react with the peroxycarboxylic acids. This group includes dimethyl sulfoxide, carbon disulfide, and solvents which contain multiple bonds. Also, it is desirable that the organic co-solvent selected not solubilize the peroxycarboxylate product to a significant extent. This will facilitate the isolation of the product as a solid precipitate. The precipitate can be separated by any standard technique. For example, filtration, decantation, and/or centrifugation can be used. The precipitate can then be washed, for example, with water, and if used, an organic co-solvent, to remove any unreacted starting materials. Finally, the precipitate is dried to remove excess water. This may be achieved by any standard means of drying. For example, vacuum desiccation, mild heating at ambient pressure, or air drying may be used.

In some embodiments the salt is a basic metal salt. In some embodiments of the invention, the amount of basic metal salt added to the reaction mixture is sufficient to adjust the pH of the reaction solution to, from about 2 to about 8, preferably from about 3.5 to about 7.5, more preferably from about 3.8 to about 7, and most preferably less than 5. Lower pHs are preferred as the peroxycarboxylic acid may rapidly destruct at high pH, which undesirably reduces the amount of retained peroxycarboxylic acid. In a preferred embodiment, the pH of the reaction solution is lower than the pKa of the peroxycarboxylic acid. For example, in one embodiment, the reaction may take place at a pH of from about 3.8 to about 7.5, when using peroxyacetic acid, which has a pKa of 8.2. The molar ratio of peroxycarboxylic acid to metal salt may vary, depending on which salt is being used. The preferred ratio is that which enables essentially all of the metal and peroxycarboxylic acid to interact, and essentially none of each compound to be wasted. In another embodiment of the invention, the amount of metal from the metal salt, and the amount of peroxycarboxylic acid utilized in the reaction solution or suspension, is such that the molar ratio of metal to peroxycarboxylic acid is from about 0.01:1 to about 10:1. In a different embodiment of the invention, the molar ratio of metal to peroxycarboxylic acid is from about 0.5:1 to about 8:1. In another embodiment of the invention the molar ratio of basic metal salt to peroxycarboxylic acid is from about 0.01:1 to about 0.5:1. In another embodiment of the invention the molar ratio of basic metal salt to peroxycarboxylic acid is no more than 0.5:1. In another embodiment of the invention the molar ratio of basic metal salt to peroxycarboxylic acid is about 6:1. In yet another embodiment of the invention the molar ratio of metal to peroxycarboxylic acid is about 1.5:1. In still another embodiment of the invention, the molar ratio of metal to peroxycarboxylic acid is about 0.7:1. In a further embodiment of the invention, the antimicrobial composition contains from about 0.1 wt % to about 85 wt % of the peroxycarboxylic acid.

Coatings of the composition according to the present invention have a tendency to quickly lose their antimicrobial activity over time, which is believed to be the result of evaporation of the neat peracid. The salt in the composition forms a salt of the peracid, which testing has shown to retain antimicrobial activity over a lengthy accelerated aging test. The salts in the composition serve to stabilize the antimicrobial composition. It is well known in the art that peroxycarboxylic acids are highly volatile due to their high vapor pressures. They are also known to readily lose their active oxygen, making them unstable. By “active oxygen” is meant the oxygen contained in a molecule that is easily transferred via a chemical reaction to another compound. Peroxycarboxylic acids tend to be even more unstable when mixed with other compounds. As a result, compositions containing peroxycarboxylic acids have poor storage stability. Previous attempts to provide antimicrobial solutions containing hydrogen peroxide and peroxycarboxylic acid were unsuccessful in the absence of the basic metal salt, due in large part, to loss, during the drying process, of most of the peroxycarboxylic acid from the formulation, due to volatilization. In addition, the liquid antimicrobial composition suffered comparable losses of hydrogen peroxide, resulting in a final antimicrobial composition that did not have the desired antimicrobial properties. The formation of the solid-phase peroxycarboxylate metal salt overcomes the challenge of maintaining the volatile components of the antimicrobial composition, producing a dry antimicrobial composition having the desired properties. The addition of basic metal salts to the antimicrobial composition is thus useful for achieving a quantifiable amount of peroxycarboxylic acid in the antimicrobial composition, even though the more volatile species are still lost. The peroxycarboxylate moiety of the antimicrobial composition's peroxycarboxylic acid associates with the metal in the basic metal salt in such a way that provides stability to the solid-phase peroxycarboxylic acid metal salt. In other words, the active oxygen in the metal peroxycarboxylate is retained during storage to a greater extent than is the active oxygen contained in the corresponding peroxycarboxylic acid. Also, the solid-phase peroxycarboxylic acid metal salts have superior odor, dispersibility and handling properties relative to the corresponding peroxycarboxylic acids. The long-term stability of the solid-phase peroxycarboxylic acid metal salt may be negatively affected by the presence of water. Therefore, during drying of the solid-phase peroxycarboxylic acid metal salt precipitate, it is desirable to eliminate as much of the solution-phase water as possible. In a preferred embodiment, only waters of crystallization are present in the solid-phase peroxycarboxylic acid metal salt. In another preferred embodiment, the solid-phase peroxycarboxylic acid metal salt contains no more than 12 water molecules per metal ion, with the amount of acceptable water molecules depending on the peroxycarboxylic acid being used. Suitable salts include, for example, a lithium, sodium, potassium, rubidium, cesium, zinc salt, magnesium, or calcium salt, or any combination thereof. In some embodiments, the salt is a magnesium salt, such as for example a magnesium oxide, magnesium hydroxide, magnesium carbonate, magnesium acetate tetrahydrate, and a combination thereof. In some embodiments, the salt is magnesium acetate tetrahydrate.

In some embodiments of the invention, the reaction solution or suspension can also contain other additives. For example, in some embodiments of the invention, the reaction solution or suspension can also include a polyethylene glycol (PEG). Any type of polyethylene glycol may be used for the invention, whether a low molecular weight PEG, such as for example, PEG 200, PEG 300 or PEG 400, a high molecular weight PEG, such as for example PEG 3350, PEG 4000, PEG 10,000 or PEG 35,000, or a medium molecular weight PEG. The amount of polyethylene glycol will depend on a variety of factors, such as for example, the application or the type of peroxycarboxylic acid. In some embodiments of the invention, the polyethylene glycol is present in the reaction mixture in an amount of from greater than 0 to 75 weight percent, based on the total weight of the reaction mixture.

The stability of the compositions may also be affected by the humidity under compositions are stored. In some embodiments of the invention, the compositions are stored under conditions where the relative humidity is less than about 43, preferably less than about 40, more preferably less than about 35.

For the purpose of this invention a “stable” composition is one which maintains sufficient physical properties and active oxygen content long enough to be useful, about twelve months.

The composition of the present invention have utility in numerous household products. The present invention thus also provides an antimicrobial product containing the compositions of the present invention. In some embodiments, the product is a household care product. Within such embodiments, in some cases the house hold care product is selected from hard surface cleaners, deodorizers, fabric care compositions, fabric cleaning compositions, manual dish detergents, automatic dish detergents, floor waxes, kitchen cleaners, bathroom cleaners, and combinations thereof. In other embodiments, the antimicrobial product is selected from hard surface cleaners, deodorizers, fabric care compositions, fabric cleaning composi-tions, manual dish detergents, automatic dish detergents, floor waxes, kitchen cleaners, bathroom cleaners, and combinations thereof. Antimicrobial products of the invention can be used in a wide variety of settings including, but not limited to, in health care facilities such as hospitals, rehabilitation, assisted living facilities, etc.

In other embodiments, the antimicrobial product is a medical device disinfectant. Still in other embodiments, the antimicrobial product is used as a disinfectant for aseptic filling equipment. Yet in other embodiments, the antimicrobial product is used in an aseptic food processing system. In other embodiments, the antimicrobial product is used as a disinfectant for biofilms in water systems. Still in other embodiments, the antimicrobial product is used as a disinfectant for waste water treatment.

In some embodiments, the composition is a wound healing composition. In some embodiments, the wound healing composition is formulated as a gel, a liquid, lotion, skin patch, irrigation gel, a spray, application granules, or a combination thereof for healing wounds.

Studies show that many widely used wound antiseptics have undesired cytotoxicity, and while some do kill bacteria at a sufficient level, they often do not promote a relatively fast wound healing. In many cases, irrigation of open fracture wounds with an antibiotic solution offers no significant advantages over the use of a nonsterile soap solution and may in fact increase wound-healing problems.

To be useful, topical antiseptics should be toxic to bacteria but should have no significant toxicity to underlying tissues, and ideally, they should also preserve or enhance host defense against infection. The present invention also provides a method for treating wounds including, but not limited to, surgical wounds, battle wounds, accidental wounds, thermal burn wounds, chemical burn wounds, chronic wounds, decubitus ulcesr, foot ulcers, venous ulcers, laser treatment wounds, sunburns, or abrasions. In some embodimens, the infected wound is contacted with the composition daily. Methods of the invention promote wound healing and typically rapidly kill high levels of viruses, vegetative bacteria, fungi, mycobacteria and spores. Unlike many conventional antiseptics available today, compositions and methods of the invention eliminate bacteria, enhance body's defense system, and enhance the healing process.

In addition, the combination of the compositions disclosed in the present embodiments can kill high levels of bacteria and spores in biofilms and in high protein environments without being corrosive and having virtually no cellular toxicity issues.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

### EXAMPLES

The present invention is described more fully by way of the following non-limiting examples. Modifications of the examples will be apparent to those skilled in the art.

### Example 1

Compositions capable of forming shelf-stable coatings containing the magnesium salt of peroxyacetic acid were prepared by drying solutions containing a magnesium salt, acetic acid, hydrogen peroxide, peracetic acid, and poly(ethylene glycol) (PEG). The starting magnesium salt was magnesium hydroxide, magnesium carbonate, or magnesium acetate tetrahydrate (an anhydrous magnesium acetate salt would also be effective since it is being dissolved in a water-containing mixture). The acetic acid/hydrogen peroxide/peracid source was an aqueous solution (called “PAA Source” in this document) usually containing 8-12 wt % peracid (peracetic acid), 15-22 wt % hydrogen peroxide, and 14-20 wt % acetic acid. Coatings were also be made in the presence of silica particles (up to 2.8%). Finally, the remainder of the solution typically consisted of water, but the short-chain alcohols methanol, ethanol, and isopropanol were also successfully used, with the shortest chains being the most successful.

A typical coating-solution mixture consisted of the following, which was used immediately after mixing:

4.1 wt % magnesium acetate tetrahydrate

### 20 wt % PAA Source

### 20 wt % PEG 3350

55.9 wt % water

Magnesium acetate tetrahydrate concentrations in the 1.8-6.5 wt % range were used successfully, with the best peracid recoveries occurring at higher concentrations. PAA Source concentrations of 8-72 wt % were found to yield stable peracid salts. PEG concentrations of 0-30 wt % were tested successfully. PEG 3350 and PEG 8000 both yielded coatings containing stable peracid salts.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and mod-ifications are within the scope of the invention, e.g., as may be within the skill and knowledge of those in the art, after understanding the present disclosure. It is intended to obtain rights that include alternative embodiments to the extent permitted, including alternate, interchange-able and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A solid state antimicrobial composition comprising
a peroxyacid, a hydroperoxide, the parent carboxylic acid of the peroxyacid, and a salt selected from the group consisting of a lithium salt, a sodium salt, a potassium salt, a rubidium salt, a cesium salt, a zinc salt, a magnesium salt, a calcium salt and a combination thereof.

2. The composition of claim 1, wherein the hydroperoxide is hydrogen peroxide.

3. The composition of claim 1, wherein the salt is a magnesium salt.

4. The composition of claim 3, wherein the magnesium salt is selected from the group consisting of a magnesium oxide, magnesium hydroxide, magnesium carbonate, magnesium acetate tetrahydrate, and a combination thereof.

5. The composition of claim 3, wherein the magnesium salt is magnesium acetate tetrahydrate.

6. The composition of claim 1, wherein the peroxyacid is peracetic acid.

7. The composition of claim 1, wherein the peroxyacid comprises a magnesium salt of the peroxyacid.

8. The composition of claim 1, wherein the composition further comprises at least one of a bis(hydroperoxide) or an epoxide.

9. The composition of claim 8, wherein the peroxyacid is peracetic acid and the bis(hydroperoxide) is 3,3-bis(hydroperoxy)butanoic acid.

10. The composition of claim 1 formulated as a wound healing composition.

11. The composition of claim 1, wherein the wound healing composition is formulated as a gel, a liquid, lotion, skin patch, irrigation gel, a spray, application granules, or a combination thereof for healing wounds.

12. A method for treating a wound infection in a subject comprising contacting the infected wound in the subject with a therapeutically effective amount of the composition of claim 10.

13. The method of claim 12, wherein the infected wound is a surgical wound, battle wound, accidental wound, thermal burn wound, chemical burn wound, chronic wound, decubitus ulcer, foot ulcer, venous ulcer, laser treatment wound, sunburn, or an abrasion.

14. The method of claim 12, wherein the infected wound is contacted with the composition once a day.

15. The method of claim 12, wherein the composition is formulated as a gel, a liquid, lotion, skin patch, irrigation gel, a spray, application granules, or a combination thereof.
